(19) United States
(12) Patent Application Publication
(10) Pub. No.: US 2025/0082352 A1
(43) Pub. Date: Mar. 13, 2025

(54) TONGUE SCRAPER WITH SUCTION

(71) Applicant: Dale Medical Products, Inc., Franklin, MA (US)

(72) Inventors: Melissa SCHULTZ, Crown Point, IN (US); Ronald D. RUSSO, Naples, FL (US)

(21) Appl. No.: 18/957,405

(22) Filed: Nov. 22, 2024

Related U.S. Application Data
(63) Continuation-in-part of application No. 18/657,372, filed on May 7, 2024.
(60) Provisional application No. 63/500,990, filed on May 9, 2023.

(51) Int. Cl. A61B 17/24

(52) U.S. Cl. CPC A61B 17/244

(57) ABSTRACT

A manually operated tongue scraper with suction application assist is disclosed. The scraper is especially useful in medical applications where patients have accumulated thick viscous tongue secretions and biofilm due to dry mouth and oxygen therapy. The scraper has a tubular body with a distal end and a proximal end and a flow path suction channel transversing between the distal and proximal ends of the tubular body. The distal end having a scraper head portion with a scraping edge element and the head including a suction lumen accessing said flow path suction channel. The flow path suction channel removing accumulated tongue secretions and biofilm from the scraper head portion when suction is applied to the proximal end of the scraper.

### CROSS-REFERENCE TO RELATED APPLICATION

This application is a continuation-in-part application of U.S. patent application Ser. No. 18/657,372 filed May 7, 2024, which claims priority to claims priority to U.S. Patent Provisional Application No. 63/500,990, filed May 9, 2023, the entire contents of each of which are hereby incorporated by reference.

### BACKGROUND OF THE INVENTION

Tongue scraping of accumulated biofilm on the tongue is generally considered good oral hygiene. Various scraper types are commercially available consisting of curved headed plastic molded instruments with molded in handles. The scraping edges on the curved headed sections are generally sharp edges or multiple rows of built in edges. The scraped biofilm is usually manually scraped off the tongue and deposited in a bathroom sink and the mouth then rinsed with water. Removal of this biofilm of food debris, dead cells and bacteria results in better breath and a cleaner oral cavity. This is especially true of hospital patients, particularly those that are heavily sedated, comatose, or being managed with an advanced airway. Those patients tend to have very thick accumulated tongue biofilm from dry mouth and from the constant administration of oxygen. Further, this accumulation of tongue biofilm has been associated with contributing to ventilator associated pneumonia (VAP). However, it is very difficult to perform tongue scraping on a bedridden patient such that most hospital protocols simply require toothbrushing and oral secretion suctioning using a separate Yankauer suction instrument. As such, oral tongue scraping is not usually performed due to the difficulty involved with hospitalized patients.

It would be especially beneficial and useful to provide a single patient use tongue scraper that could remove the thick, viscous biofilm from a patient's oral cavity to help reduce the likelihood of VAP.

### SUMMARY OF THE INVENTION

The manually operated tongue scraper with suction application assist is configured in a one-piece unitized injection molded rigid plastic construction. The scraper has a tubular body with a distal end and a proximal end. A flow path suction channel transverses between the distal end and the proximal end of the tubular body. The distal end has a scraper head portion with a molded in scraper edge. The scraper head portion also includes a suction lumen which accesses the flow path suction channel housed within the tubular body. The proximal end of the tubular body includes a molded-in barbed fitting for connection to a suction tubing line. The flow path suction channel removes accumulated tongue biofilm and secretions when biofilm and secretions are scraped from the tongue and suction is applied to the proximal end of the tubular body during and after scraping activity.

### BRIEF DESCRIPTION OF THE DRAWINGS

FIG. 1 is a partial cross sectional perspective view of the present invention scraper connected to a suction tubing line.

FIG. 2 is partial cross sectional side view of the tongue scraper with suction.

FIG. 3 is a cross sectional frontal view of the scraper head portion of the present invention taken along lines A-A from FIG. 2

FIG. 4 is a partial bottom view of the scraper head portion of the present invention.

FIG. 5 is a partial cross sectional side view of the scraper head portion of the present invention taken along lines B-B from FIG. 4.

FIG. 6 is a partial top side view of the scraper head portion of the present invention.

FIG. 7 is a partial side view of the scraper head portion of the present invention.

FIG. 8 is a partial cross sectional view of the scraper, head portion scraping biofilm from the tongue and the biofilm being directed into a suction lumen by a scraping edge element and out through the flow path suction channel.

FIG. 9 is a partial cross sectional view of an alternate embodiment of the scraper head portion of the present invention depicting both distal end and bottom side suction lumens.

FIG. 10 is a cross sectional frontal view of an alternate embodiment of the scraper head portion of the present invention depicting the scraping edges with molded in saw teeth elements.

FIG. 11 is a partial cross sectional perspective view of an alternate embodiment of the tongue scraper with suction having a suction control regulator valve incorporated within the body of the scraper.

FIG. 12 is a partial cross sectional perspective view of an alternate embodiment of the tongue scraper with suction having a suction control regulator valve as part of an extended handle with the addition of oral brushing elements incorporated as part of the scraper head portion.

FIG. 13 is a partial perspective view of another alternative embodiment of the scraper having an oral cleansing sponge element as part of the scraper head portion.

### DETAILED DESCRIPTION OF THE INVENTION

FIG. 1 is a partial cross sectional perspective view of the tongue scraper with suction 10 of the present invention. Scraper 10 comprises a one piece unitized injection molded rigid plastic construction forming a tubular handle type body 11 with a distal end 12 and a proximal end 13. Typically, the rigid plastic material used in its molding is clear blue tinted K-resin or high impact styrene. A flow path suction channel 14 partially shown fully transverses between distal end 12 and proximal end 13. The full length of the scraper 10 is about 6 inches in length and the flow path suction channel 14 transverses that full length. The outside diameter 15 of tubular body 11 is 0.265 inches and the interior flow path suction channel 14 inside diameter 16 is 0.160 inches (4 mm). Distal end 12 incorporates a scraper head portion 17 having a frontal curved scraper configuration edge 18 which tapers back 19 into tubular body 11. Tubular body 11 further extends 20 into scraper head portion 17, as does flow path suction channel 14.

Proximal end 13 includes a molded in barbed fitting 21 for connection to a suction tubing line 22. If the scraper 10 is utilized on a hospitalized patient, then suction is supplied through hospital supplied suction tubing 22. Often, hospital supplied suction is supplied using wall cannistered suction (not shown) which acts as a reservoir for accumulated suctioned fluid. The scraper is not limited to a hospital setting, but is also utilizable in a home health, dental, nursing home, or home setting. Barbed fitting 21 permits connection to any type of external suction source such as portable, pump, or compressor.

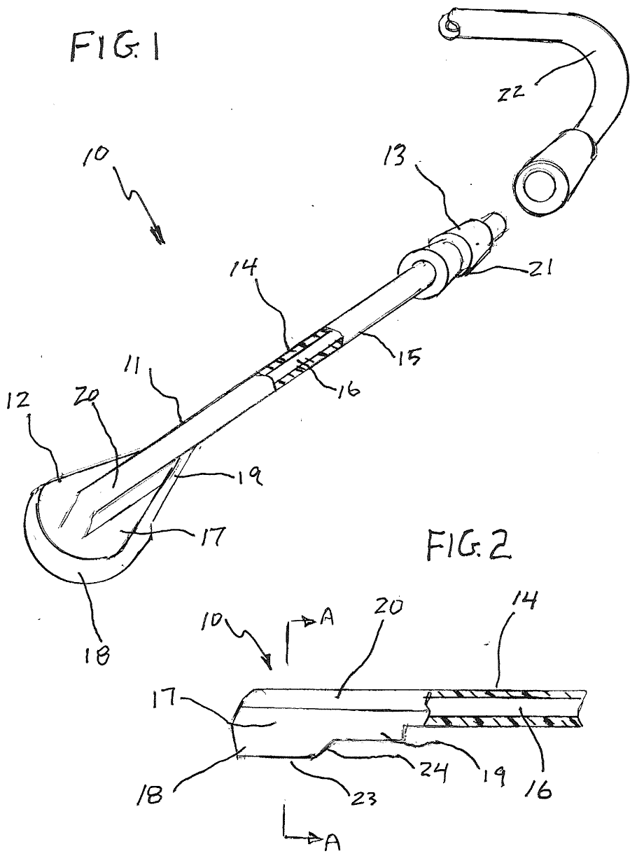

FIG. 2 is a partial cross sectional side view of scraper 10 clearly depicting flow path suction channel 14 with a inside flow path diameter 16 wherein flow path diameter 16 further extends 20 into scraper head portion 17. Scraper edge 18 extends rearward on scraper head portion 17 along scraper surface 23 into angled edge 24 which terminates in tapered back 19.

Before tongue scraping with suction can begin on a patient, the clinician will first suction the mouth using a Yankauer suction tube to remove pooled saliva and secretions. The suction level is usually set at 150 mm Hg. The clinician next disconnects the Yankauer from the suction tubing line and attaches scraper 10 to the suction tubing line 22. The suction remains at the 150 mm HG level. Scraping begins from the back of the tongue to the front while maintaining the tongue scraping with suction action provided by scraper 10 of the present invention. A mouth moisturizing oral gel containing glycerin may also be applied to the surface of the tongue in order to loosen the encrusted tongue biofilm.

The manually operated tongue scraping with suction application assist helps to remove the thick viscous biofilm from the patient's tongue. After scraping, the scraper is usually flushed and cleaned by inserting scraper head portion 17 into a plastic hospital bowl usually filled with 100 ml of warm water to flush the scraper 10 and tubing line 22. After use, scraper 10 usually is stored in its plastic bowl and can be used repeatedly for 24 hours before disposal. In effect, scraper 10 is a single patient use, non-sterile product that should be replaced after 24 hours of use. However, it can also be routinely used as a single scraping procedure product and disposed of after each scraping procedure due to its low cost plastic injection molded process.

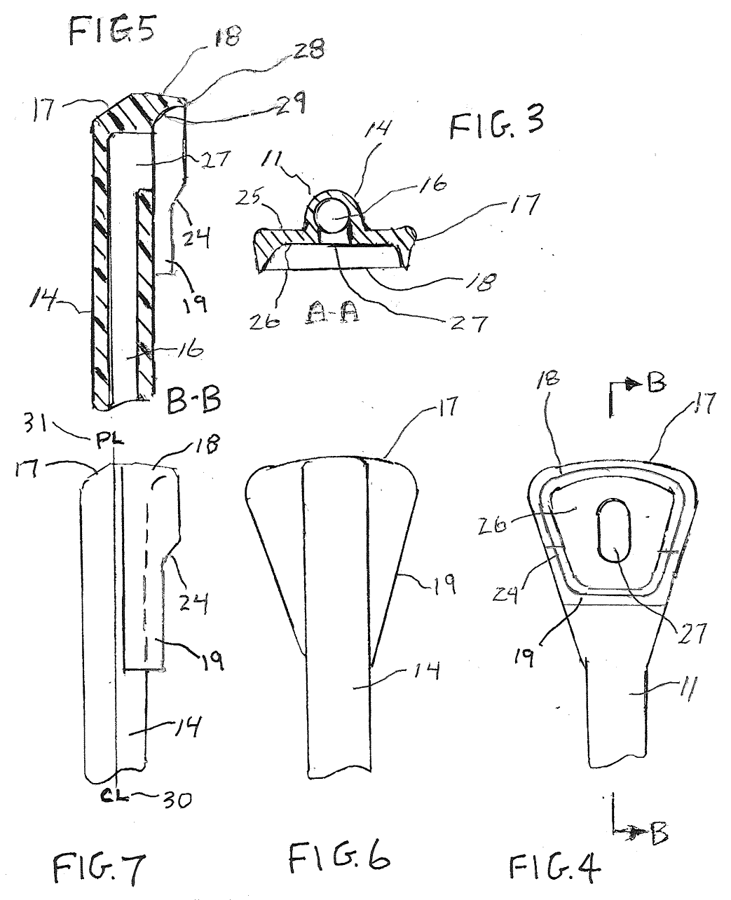

FIG. 3 is a cross sectional frontal view of scraper 10 taken along lines A-A from scraper side view from FIG. 2. The tubular body 11 with flow path suction channel 14 with inside 4 mm diameter 16 is clearly depicted. Scraper head portion 17 with scraper edge 18 is also clearly depicted. Importantly, scraper head portion 17 has a top side 25 and a bottom side 26. The bottom side 26 includes bottom edge 18. The bottom side 26 has a suction lumen 27 which accesses flow path suction channel 14 and inside diameter 16 which permits suctioned biofilm secretions removed by scraper edge 18 to be suctioned through suction lumen 27 back into flow path suction channel 14 and inside diameter 16.

FIG. 4 is a partial bottom side view of scraper head portion 17 depicting scraper edge 18 which is curved and tapers back into angled edge 24. Angled edge 24 completely surrounds bottom side 26 such that scraped secretions are completely retained within bottom side 26 and can be suctioned out suction lumen 27. The lower portion of angled edge 24 also prevents scraped biofilm from dribbling down handle type body 11. Scraper edge 18 continues around the entire periphery of scraper head portion 17 including tapered back 19. Bottom side 26 suction lumen 27 is configured in an extra large oval flow path configuration of a width of 4 mm and a length of 6 mm. This is also shown in partial cross sectional side view FIG. 5 which is taken along lines B-B from FIG. 4. Scraper edge 18 is 3 mm wide and 3 mm in height with a pointed scraping edge element 28 of 0.5 mm in curvature. Backside curvature 29 of element 28 has a curved radius of 1/16 inches. For maximum biofilm removal scraping edge element 28 is positioned in front of suction lumen 27 while backside curvature 29 further helps element 28 to funnel and direct scraped biofilm into lumen 27 and into flow path channel 14.

FIG. 6 is a partial top side view of curved scraper head portion 17 which is 0.750 inches in width and one inch long at its tapered back 19. Flow path suction channel 14 is positioned on top of scraper head portion 17.

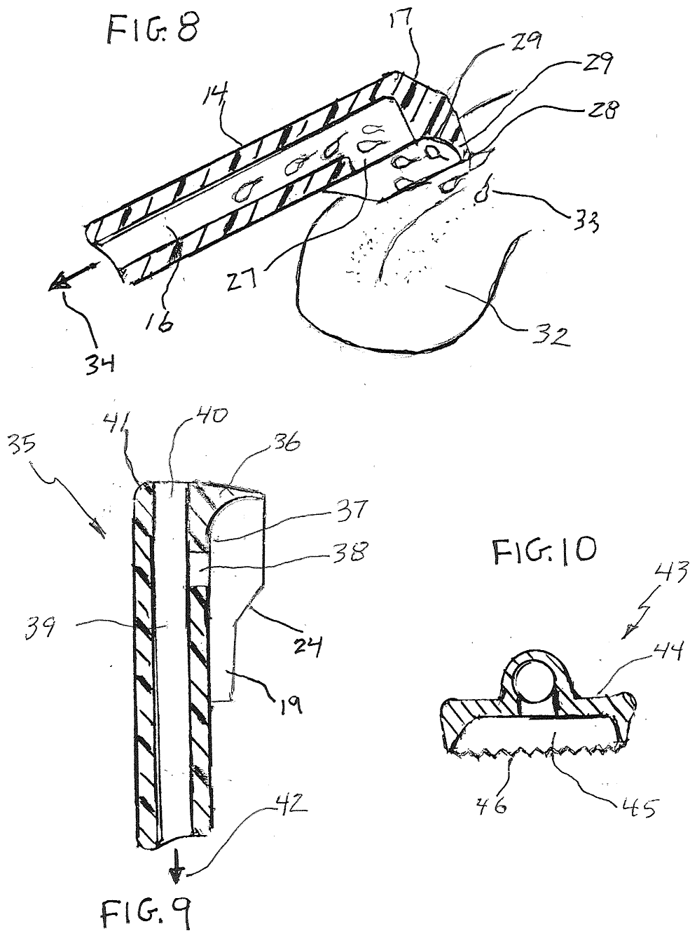

FIG. 7 depicts a partial side view of scraper head 17 illustrating both the center line 30 (CL) and parting line 31 (PL) of scraper 10 when injection molded in one piece.

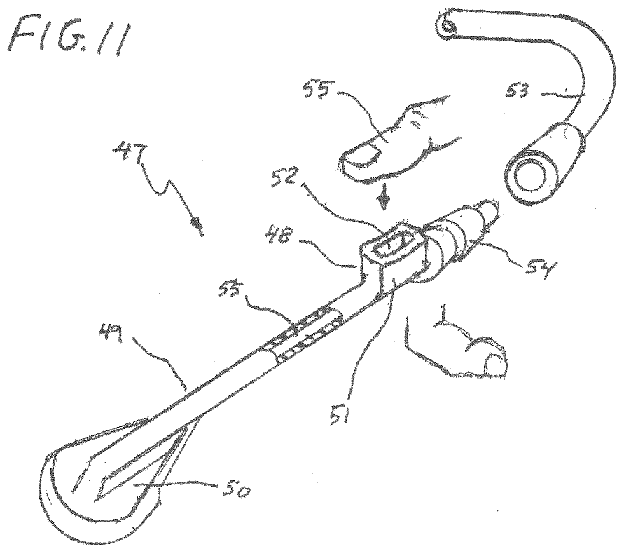

FIG. 8 is a partial cross sectional view of the scraper 10 depicting the scraper head portion 17 tongue scraping the tongue 32 of accumulated biofilm 33. Scraping edge element 28 on scraper head portion 17 is aided by its underside curvature 29 to funnel and direct biofilm secretions 33 into bottom side 26 suction lumen 27 which in turn is suctioned off 34 through flow path suction channel 14 using hospital wall suction into internal inside diameter 16. It has been determined that scraping by itself is not powerful enough to remove the thick viscous hospital patient accumulated biofilm from the mouth. The assistance of suction power is required as illustrated and described in FIG. 8. This is especially true in patients being managed with an advanced airway. As such, the scraper 10 as depicted and described in both FIG. 8 and FIG. 1 illustrate the closed aspiration suctioning of potentially infectious biofilm through suction channel 14 and out through suction tubing as illustrated from FIG. 1. This closed suction oral aspiration suctioning prevents the spraying and depositing of potentially infectious material in and around the patient's clothing, gown, bedsheets, and breathing circuitry to prevent disease spreading as well as protecting clinicians from contacting this material.

Closed system tongue scraping with suction removes the accumulated biofilm secretions and reduces the level of oral bacteria. Removal of this biofilm also reduces accumulation of the biofilm bacteria on the airway tube which helps to reduce the likelihood of ventilated associated pneumonia (VAP).

FIG. 9 illustrates an alternate embodiment of scraper 35 which has a similar scraper head portion 36 to previously shown and described scraper head portion 17 from FIGS. 4 and 5. What is different in scraper head portion 36 from FIG. 9 is that it now has an underside 37 with a 4 mm suction lumen 38 that accesses flow path suction channel 39 that terminates in an additional extended flow path suction channel opening 40 of 4 mm internal diameter. As such, alternate embodiment scraper 35 now has two suction head lumens comprising underside suction lumen 39 and extended channel opening 40. This alternate embodiment provides the dual purpose of tongue biofilm suction removal through underside 37 lumen 38 while extended opening 40 provides the suction ability to remove oral saliva secretions through its distal tip end 41 channel opening 40. The single hospital supplied suction source 42 will apply suction through both lumens 38 and 40 simultaneously.

FIG. 10 illustrates a cross sectional view of another alternate embodiment 43 of scraper head portion 44 very similar to scraper head portion 17 illustrated and described in FIG. 4. What is different is that scraper edge 45 now has a series of mini sawtooth teeth 46 which are 0.5 mm wide by 0.5 mm high. These mini teeth 46 may aid in more effective scraping of thick viscous biofilm. Some clinicians may prefer the scraping action provided by the added mini teeth 46 as shown in FIG. 10.

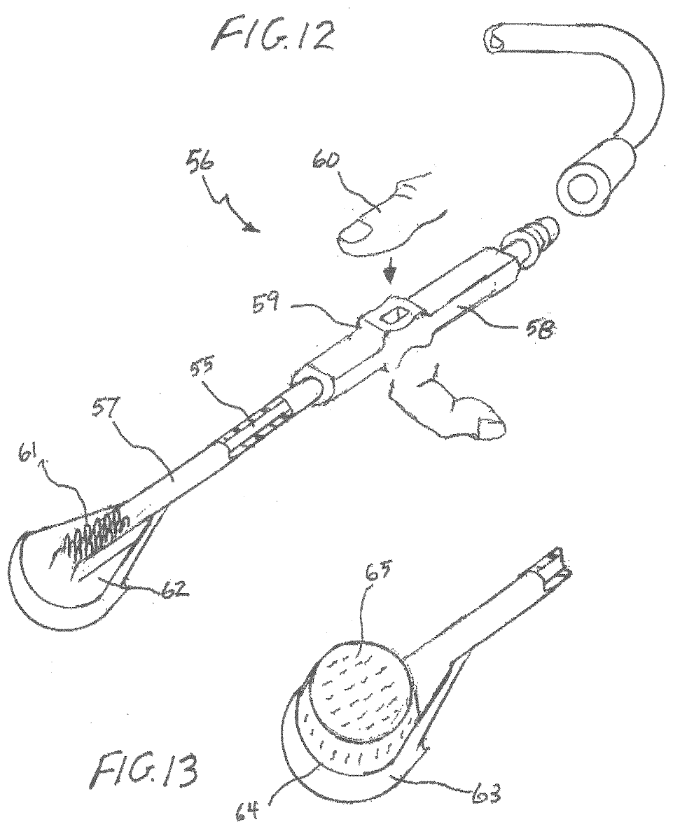

FIG. 11 is a partial cross sectional perspective view of another alternate embodiment of the tongue scraper with suction 47 having a suction control regulator valve 48 incorporated within the body 49 of the scraper 47. The balance of the structure of the scraper 47 is identical to the structure of scraper 10 shown and described in FIG. 1.

Valve 48 is used to control and regulate suction being applied to scraper head portion 50.

Valve 48 comprises a raised stem chimney 51 incorporated as part of body 49. Stem chimney 51 has an open valve port 52 which is open to atmosphere. When suction is applied via suction tubing 53 through suction connector end 54, the applied suction is vented to atmosphere through valve open port 52. As such, no suction is applied through interior flow path suction channel 55 and applied suction to scraper head portion 50 is blocked. Valve 48 is manually operated by thumb pressure 55. Thumb pressure 55 can control the application of suction to the scraper head portion 50 by closing off port 52. Full thumb pressure will apply full suction application while the degree of suction can be controlled by partially closing off port 52. In this manner, the degree and level of suction being applied to a patient's tongue during suction scraper action is now controlled and regulated by use of valve 48.

Likewise, FIG. 12 depicts another alternate embodiment of tongue scraper with suction 56. The embodiment 56 shown in FIG. 12 functions and operates in an identical manner to the scraper 47 shown and described in FIG. 11 with two major variations. The first variation is the body 57 incorporates an extended handle portion 58 having a manually operated suction control regulator valve 59. Valve 59 operates and functions just like the valve 48 shown and described in FIG. 11 by use of manually operated thumb pressure 60. Extended handle portion 58 makes it easier to manipulate and control valve 59 during tongue scraping action. In FIG. 11, the valve 48 is positioned adjacent to the proximal connector end 54, whereas the valve 59 shown in FIG. 12 is incorporated as part of extended handle portion 59.

The second major variation is the incorporation of brush elements 61 as part of scraper head portion 62. Brush elements 61 are formed in multiple rows as shown. They can be molded in as part of body 57 or separately insert molded as semi flexible bristle type elements. If molded in, both the body 57 and brush elements 61 can be injection molded from semi rigid synthetic rubber such as Santoprene®. If insert molded, then the body 57 remains as rigid plastic and the brush elements are formed of more flexible synthetic rubber or nylon. Typically, the brush elements 61 are about 1 mm in diameter and about 5 mm high and are incorporated as part of scraper head 62. The purpose of brush elements 61 is to break up and loosen encrusted biofilm on the tongue for easier removal with the tongue scraper. The brush elements should be used with either an oral rinse or water which may assist in softening encrusted biofilm and reducing friction.

FIG. 13 is a partial perspective view of another alternate embodiment of the scraper head portion 63 having a flat top surface 64. Adhesive bonded onto top surface 64 is a circular open cell synthetic polyurethane formed sponge element 65 about 5 mm high in thickness. The purpose of sponge element 65 is to help moisten encrusted biofilm for easier removal with the tongue scraper with the use of oral rinse or water prior to tongue scraping. At the conclusion of scraping, the sponge can be used with or without rinse or water to remove any excess biofilm. While sponge element 65 is shown in a circular shape, it can easily be formed in other shapes such as rectangular.

As shown and described in the drawings and detailed description of the present invention, the tongue scraper with suction provides a useful and unique instrument for better oral care especially in hospitalized patients.

Various additions or modifications can be made to the overall design of the device without departing from the underlying novelty of the shown and described inventions. For instance, a slight molded-in texture may be molded into the tubular body handle 14 to aid in manual gripping of the tubular body handle 14 illustrated in FIG. 1 without effecting the overall performance of the described invention. Likewise, various modifications to the design of the scraper head portion of the device can be made without departing from the uniqueness of the underlying invention as shown and described in alternate embodiments from FIGS. 9 and 10.

Different types of scraper head configurations can be easily substituted without departing from the novelty of the present invention and the invention as claimed.

1. A tongue scraper with suction application assist for removing manually scraped biofilm comprising;
a tongue scraper body having a distal end and a proximal end, said body including a flow path suction channel transversing between said distal end and said proximal end, said distal end configured with a scraper head portion with a suction lumen accessing said body flow path channel for removing scraped oral biofilm, said scraper head portion further having a scraping edge element, said scraping edge element directing scraped oral biofilm into said scraper head suction lumen when said scraper is manually operated and oral biofilm removed through said flow path suction channel when suction application is applied to the proximal end of said tongue scraper body, wherein a suction control regulator valve is incorporated as part of the body of the tongue scraper.

2. The scraper of claim 1 wherein the scraper edge element is positioned in front of the scraper head suction lumen.

3. The scraper of claim 1 wherein suction is applied to a hospital patient's oral cavity utilizing hospital supplied suction through suction tubing.

4. The scraper of claim 1 wherein the scraper is connectable at its proximal end to any type of external suction source.

5. The scraper of claim 1 wherein the scraper head portion has a topside and a bottom side, said bottom side having said scraper edge element.

6. The scraper of claim 1 wherein the scraper head portion has a top side and a bottom side, said bottom side having a suction lumen.

7. The scraper of claim 1 wherein the scraper head portion removes oral secretions from a patient's tongue.

8. The scraper of claim 1 wherein the scraper head portion removes oral secretions from an intubated patient's tongue.

9. The scraper of claim 1 wherein the scraper is plastic molded in one piece.

10. The scraper of claim 1 wherein the scraper is disposable after use.

11. The scraper of claim 1 wherein the scraper is utilized in a hospital, home health, dental, nursing home, or home setting.

12. The scraper of claim 1 wherein the scraping edge element has a curved scraper configuration edge.

13. The scraper of claim 1 wherein the scraper is a single procedure product disposed of after each use.

14. The scraper of claim 1 wherein the scraper is molded in one piece of a rigid plastic.

15. The scraper of claim 1 wherein the proximal end includes a fitting for connection to suction tubing.

16. The scraper of claim 1 wherein the suction control regulator valve is positioned adjacent to the proximal connector end of the scraper.

17. The scraper of claim 16 wherein the suction control regulator valve is manually operated by thumb pressure.

18. The scraper of claim 1 wherein the body of the scraper incorporates an extended handle portion having a manually operated suction control regulator valve.

19. The scraper of claim 18 wherein the suction control regulator valve is manually operated by thumb pressure.

20. The scraper of claim 1 wherein brush elements are incorporated as part of the scraper head portion.

21. The scraper of claim 1 wherein a sponge element is incorporated as part of the head portion.
